(19) United States
(12) Patent Application Publication
(10) Pub. No.: US 2009/0099597 A1
(43) Pub. Date: Apr. 16, 2009

(54) SUTURE ASSEMBLY WITH TISSUE ENGAGING ELEMENTS

(76) Inventor: Nicanor G. Isse, Burbank, CA (US)

Correspondence Address: ROSENBAUM & ASSOCIATES, P.C., 650 DUNDEE ROAD, SUITE #380, NORTHBROOK, IL, 60062, US

(21) Appl. No.: 11/871,648

(22) Filed: Oct. 12, 2007

(51) Int. Cl. A61L 17/00; A61B 17/04

(52) U.S. Cl. 606/228

(57) ABSTRACT

Multiple embodiments of a suture assembly for use with tissue are provided. One suture assembly includes a tubular body having a plurality of tissue engaging elements formed from the tubular body. An outer surface is disposed on each tissue engaging element, and the outer surface contacts the tissue. An interior surface is disposed on each tissue engaging element, and the interior surface accepts tissue growth. A supporting member joins and separates two tissue engaging elements. Another embodiment offers a suture assembly comprising a tubular body and a plurality of tissue engaging elements are formed from the tubular body. Each tissue engaging element has an outer surface that contacts the tissue. A supporting member joins and separates two tissue engaging elements.

### BACKGROUND

Methods and devices for the closure of wounds or surgical incisions are well known in the art. Sutures are a basic form of wound closure, wherein a length of filament is introduced to tissue by a needle attached to the filament. With standard sutures, the needle guides the filament through a tissue. The ends of the suture are then tied to pull the faces of a wound together, thereby closing the wound. Other methods of closure include fasteners (such as staples, clips, and clamps) and adhesives (such as glues and tapes). Embodiments described herein relate to an improved suture.

Sutures can have a wide variety of properties. Suture materials can be either absorbable or nonabsorbable, with each type of suture material being preferred for certain applications.

Absorbable sutures provide temporary wound support until the wound heals well enough to withstand normal stress. In some instances, absorption of the suture occurs by enzymatic degradation in natural materials and by hydrolysis in synthetic materials. Hydrolysis can cause less tissue reaction than enzymatic degradation. A first stage of absorption has a linear rate, lasting for several days to weeks. The second stage is characterized by loss of suture mass and overlaps the first stage. Loss of suture mass can occur as a result of, for example, leukocytic cellular responses that remove cellular debris and suture material from a line of tissue approximation. Chemical treatments, such as chromic salts, can lengthen the absorption time.

Absorbable suture materials may be preferred for internal wound repair in situations where the sewn tissues need to be held together after healing without suture reinforcement and in situations where a nonabsorbed suture may cause tissue irritation or other adverse bodily reaction over an extended period of time. A suture material may be considered to be absorbable if it “disappears” from the sewn tissue within about a year after surgery. However, many absorbable suture materials “disappear” within a shorter period of time.

Some previously available absorbable suture materials are natural and included materials such as catgut, chromic catgut, extruded collagenous materials and the like. It is projected that some absorbable sutures may be derived from synthetic polymers, which are strong, dimensionally uniform, and storage-stable in dry state. Some examples of such absorbable synthetic polymers include polyglactin, poliglecaprone, polydioxanone, lactide homopolymers, and copolymers of lactide, glycolide, and glycolide homopolymers (e.g., polyglycolic acid).

Some nonabsorbable sutures may elicit a tissue reaction that results in encapsulation of the suture material by fibroblasts. Some nonabsorbable sutures are used in percutaneous skin closures and are removed after sufficient healing has occurred. Healing may occur in about 6 to 8 days in an otherwise healthy patient. Some nonabsorbable sutures also have internal use. In some internal uses, the sutures may become encapsulated permanently in tissue. Some known nonabsorbable suture materials include, among other things, nylon, linen, silk, polypropylene, polybutester, and polyester fiber.

Sutures may be monofilament or multifilament (e.g. braided). Monofilament sutures are made of a single strand. Generally, monofilament sutures have a structure that is more resistant to harboring of microorganisms than multifilament sutures. Furthermore, monofilament sutures generally tie relatively more easily when compared with multifilament sutures. Less resistance to passage through tissue occurs with monofilament sutures than with multifilament sutures. Accordingly, great care must be taken in handling and tying a monofilament suture because crushing or crimping of this suture can nick or weaken the suture and lead to undesirable and premature suture failure.

Multifilament sutures are composed of several filaments twisted or braided together. Generally, multifilament sutures are less stiff, but they have a higher coefficient of friction than monofilament sutures. Accordingly, multifilament sutures generally have greater tensile strength and better pliability and flexibility than monofilament sutures. Generally, multifilament sutures are known to handle and to tie well. However, because multifilament materials have increased capillarity, the increased absorption of fluid may act as a tract for the introduction of pathogens.

Some times, monofilament synthetic absorbable suture materials are stiffer than their catgut or collagen counterparts. Synthetic absorbable sutures may be employed in multifilament, braided constructions in order to provide the suture with a desired degree of softness and flexibility. Such multifilament sutures may exhibit a certain degree of undesirable roughness or “grabbiness” in what has been termed as their “tie-down” performance, i.e., the ease or difficulty of sliding a knot down the suture into place.

Some of the process of suture selection depends on surgeon training and surgeon preference. A wide variety of suture materials are available for each surgical location and surgical requirement.

Aesthetic concerns are significant in the anatomic regions of the head and neck, such as the eyelid, periorbital area, nose, pinna, lip, and vermillion. In these areas, tensile strength requirements tend to be less, and smaller suture sizes are preferred. However, the mobility of the lip and vermillion requires a relatively higher suture tensile strength. The activity and mobility of the face, anterior and posterior neck, scalp, superior trunk, and nasal and oral mucosa demand higher tensile strength requirements in suture selection. Additionally, major musculocutaneous flaps tend to be closed under significant tension, requiring maximal long-term tensile strength of the suture.

Wound closure and healing is affected by the initial tissue injury caused by needle penetration and subsequent suture passage. A variety of shapes, sizes, and types of needles may be attached to a suture filament to form a suture assembly. Nonetheless, needle selection, surface characteristics of the suture (e.g., coefficient of friction), and suture-coating materials selected for wound closure are important factors that must be considered by the surgeon. Some significant surgical needle characteristics include: (1) strength for resistance of the needle against deformation during repeated passes through tissue; (2) ductility for resistance of the needle against breakage under a given amount of deformation or bending; (3) small diameter and adequate sharpness for penetration of tissue with minimal resistance; (4) sterility and corrosion-resistance to prevent introduction of microorganisms or foreign materials into the wound; and (5) clamping moment for stability of a needle in a needle holder.

Some known sutures are configured to pass only one direction through tissue, with barbs or other projections to prevent travel in the opposite direction. One example of this type of suture is presented in U.S. Pat. No. 6,241,747 issued to Ruff and U.S. Pub. No. 200710038249. These publications disclose barbed sutures or sutures with tissue-engaging elements to achieve knotless suture of wounds or surgical incisions. The '747 patent presents a barbed bodily tissue connector (i.e. barbed suture) with barbs facing both ends of the suture. The barbed suture avoids the necessity of tying knots to secure the suture, thereby reducing the time to close a wound. The use of a barbed suture also may reduce damage to tissue upon insertion of the suture and reduce scarring across the wound. However, use of this type of suture could be limited by the material composing the suture itself as the needed rigidity of the suture body could limit flexibility of the barbs. Also, care must be taken to align the barbs appropriately to reduce the chances of cutting through tissue.

The '249 patent application publication presents an elongated flexible body having a plurality of tissue-engaging elements received upon the elongated flexible body, where knots are tied in the elongated flexible body to maintain the tissue-engaging elements in serial arrangement on the elongated flexible body. One potential disadvantage of this type of suture would be the numerous individual elements comprising the suture (i.e. the elongated flexible body and the plurality of tissue-engaging elements received upon the elongated flexible body). A piece of the device could become separated from the suture within a patient's body. Also, a tissue-engaging element could be forced over a knot in the elongated flexible body, thereby interrupting the serial arrangement and reducing total engaging force for the suture.

Other uses of sutures with a textured suture strand are presented in U.S. Pat. No. 6,491,714 issued to Bennett, U.S. Pat. No. 7,033,380 issued to Schwartz et al., and U.S. Pat. No. 7,048,754 issued to Martin et al. The '714 patent discloses multiple types of protuberances that are integrally formed with a suture to enable the suture to engage an anchor element. The protuberances are not for engagement of tissue directly, but for engagement of the anchor that is attached to tissue or bone. The '380 patent shows a single bead on a suture. The bead engages a suture anchor when the bead is enclosed in a cannula of the anchor. This disclosed arrangement does not allow the bead to engage with tissue directly to assist in anchoring the suture. The '754 patent discloses a suture strand textured by suitable means to provide a surface having protuberances for securing with a ratcheting mechanism. These protuberances do not engage directly with tissue.

Importantly, the previous sutures tend to form limited, sometimes at a single location, contact with tissue. While this limited contact may be sufficient in some circumstances, it is desirable to improve the contact between the suture and tissue.

### SUMMARY

Embodiments described herein provide a suture assembly for use with tissue.

One embodiment provides a suture assembly that includes a tubular body having a plurality of tissue engaging elements formed from the tubular body. An outer surface is disposed on each tissue engaging element, and the outer surface contacts the tissue. An interior surface is disposed on each tissue engaging element, and the interior surface accepts tissue growth. A supporting member joins and separates two tissue engaging elements.

Another embodiment provides a suture assembly having a tubular body. A plurality of tissue engaging elements is formed from the tubular body and is arranged along a length of the tubular body. Each tissue engaging element has an outer surface that contacts the tissue. Each tissue engaging element has an interior surface that accepts tissue growth.

A further embodiment offers a suture assembly comprising a tubular body and a plurality of tissue engaging elements formed from the tubular body. Each tissue engaging element has an outer surface that contacts the tissue. A supporting member joins and separates two tissue engaging elements.

### BRIEF DESCRIPTION OF THE DRAWINGS

FIG. 1A is an elevational view of one embodiment of a suture assembly described herein;

FIG. 1B is an elevational view of another embodiment of a suture assembly described herein;

FIG. 2A is an elevational view of a portion of an embodiment of a suture assembly described herein;

FIG. 2B is a sectional view, taken along line B-B of FIG. 2A, showing some details of the embodiment of FIG. 2A;

FIG. 3A is an elevational view of a portion of an embodiment of a suture assembly described herein;

FIG. 3B is a sectional view, taken along line B-B of FIG. 3A, showing some details of the embodiment of FIG. 3A;

FIG. 4A is an elevational view of a portion of an embodiment of a suture assembly described herein;

FIG. 4B is a sectional view, taken along line B-B of FIG. 4A, showing some details of the embodiment of FIG. 4A;

FIG. 5A is an elevational view of a portion of an embodiment of a suture assembly described herein;

FIG. 5B is a sectional view, taken along line B-B of FIG. 5A, showing some details of the embodiment of FIG. 5A;

FIG. 6A is an elevational view of a portion of an embodiment of a suture assembly described herein;

FIG. 6B is a sectional view, taken along line B-B of FIG. 6A, showing some details of the embodiment of FIG. 6A;

FIG. 7 is an enlarged perspective view of a portion of an embodiment of the suture assembly described herein;

FIG. 8 is an enlarged perspective view of a portion of an embodiment of the suture assembly described herein; and

FIG. 9 is an enlarged perspective view of a portion of an embodiment of the suture assembly described herein.

### DETAILED DESCRIPTION

Disclosed herein are a number of embodiments of a suture assembly 10. The suture assembly 10 may be employed in a number of different applications including, but not limited to, tissue suspension, tissue approximation, tissue support, tissue fixation, wound closure and many other surgical applications. While different characteristics, materials, dimensions and compositions of embodiments of the suture assembly 10 are disclosed, it is to be appreciated that additional combinations, dimensions and materials can be used to derive a suture assembly 10 that meets requirements of a particular application.

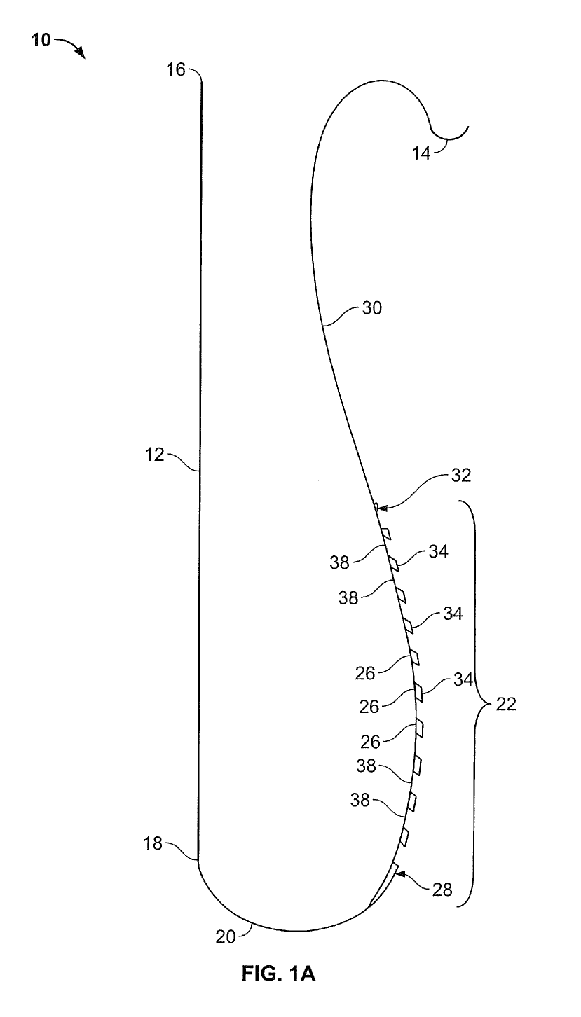

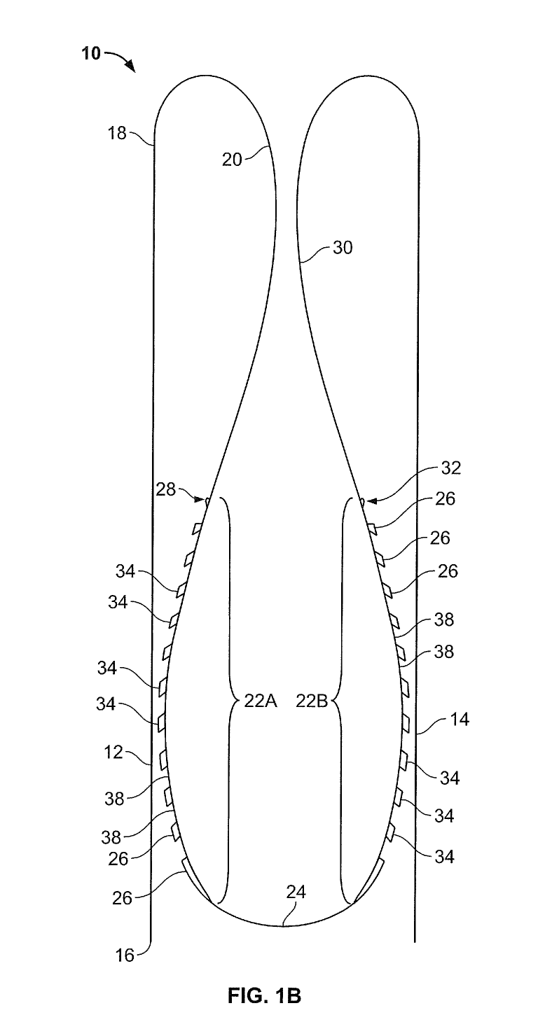

One embodiment of the suture assembly 10 is shown in FIG. 1A. This suture assembly 10 generally comprises a first member 12 and a second member 14. In one embodiment, the first member 12 may be a needle or other structure for introducing the suture assembly 10 to tissue, and the second member 14 may be a needle or other structure for facilitating tying of the suture assembly 10 to tissue. In an exemplary embodiment, the first member 12 is about 6.56 inches long. Either or both the first member 12 and the second member 14 may be substantially linear, as shown in FIG. 1B, or curved, as shown for second member 14 in FIG. 1A. Other configurations of the first member 12 and the second member 14 are possible.

A proximal end 16 of the first member 12 is constructed to facilitate insertion of the first member 12 into tissue and progress of the first member 12 through tissue. A distal end 18 of the first member 12 is connected with a first filament 20. In some embodiments, a connector having a length of about 6 mm, a width of about 0.9 mm and a thickness of about 0.13 mm joins the first filament 20 to the first member 12. The first filament 20 may comprise a monofilament suture material or a multifilament suture material, either of which may be absorbable or non-absorbable, as disclosed above in the Background section. Optionally, the first filament 20 may be solid or hollow. In am exemplary embodiment, the first filament 20 is about 2 to 3 cm long having a thickness of about 0.13 mm, a base width of about 1.27 mm, and a base opening of about 1.02 mm. In other embodiments, the first filament 20 may be substantially similar to a 3″0″ suture or a 4″0″ suture, and the constructions of those items are well known in the art. In some embodiments, a juncture between the first member 12 and the first filament 20 may be constructed to facilitate separation of the first member 12 from the first filament 20. This may be desirable upon application of the suture assembly 10 to tissue.

An end of the first filament 20 opposite to the end thereof connected with the first member 12 is coupled with an elongated, tubular body 22 shown in FIGS. 1A and 1B. In some embodiments, the body 22 may be formed from absorbable or non-absorbable materials. Some suitable absorbable materials include polylactic acid (PLA), polyglycolic acid (PGA), PLAIPGA copolymers, polycaprolactone (PCL), and the like. Some suitable non-absorbable materials include polytetrafluoroethylene (PTFE), expanded PTFE (ePTFE), and the like. The tubular body 22 is sufficiently flexible to allow introduction of the tubular body 22 into tissue following the first member 12. The tubular body 22 has sufficient structural rigidity to satisfy requirements of the particular utilization of the suture assembly 10. In an exemplary embodiment, the tubular body 22 is about 10 to 12 cm long.

In the embodiment shown in FIG. 1B, the tubular body is separated into two tubular bodies 22A and 22B by a second filament 24. The construction of the second filament 24 can be substantially similar to the first filament 20. The length of the second filament 24 may be determined by the particular needs of utilization of the suture assembly 10. It is envisioned that some embodiments of the suture assembly 10 can comprise multiple lengths of tubular bodies 22A and 22B and second filaments 24 to meet specific needs, such as but not limited to cosmetic surgery.

The tubular bodies 22, 22A and 22B have substantially similar construction, hence details of the construction will be discussed using tubular body 22 to ease understanding. Any of the following details apply equally to any of the tubular bodies 22, 22A and 22B.

Drawing attention to FIG. 1A, the tubular body 22 is constructed to form a plurality of tissue engaging elements 26. Any appropriately desired number of tissue engaging elements 26 may be constructed from the tubular body 22. In an exemplary embodiment, each tissue engaging element has a length of about 2 to 3 mm. However, other lengths may be used.

The tissue engaging elements 26 are intended to remain in contact with tissue upon intended installation of the suture assembly 10 with tissue. Because each tissue engaging element 26 contacts tissue, each tissue engaging element 26 provides a zone of contact between the suture assembly 10 and the tissue. As there is a plurality of tissue engaging elements 26, there are, likewise, a plurality of zones of contact between the suture assembly 10 and the tissue. Because the tissue engaging elements 26 are arranged or distributed along a length of the tubular body 22, so are zones of contact between the suture assembly 10 and the tissue arranged or distributed along the length of the tubular body 22. This arrangement or distribution of tissue engaging elements 22, and thus, zones of contact between the suture assembly 10 and tissue, provides the suture assembly 10 with increased stability and increased tissue holding ability. The tissue engaging elements 26 are formed from the tubular body 22 in any appropriate fashion, such as etching, deformation, cutting and the like.

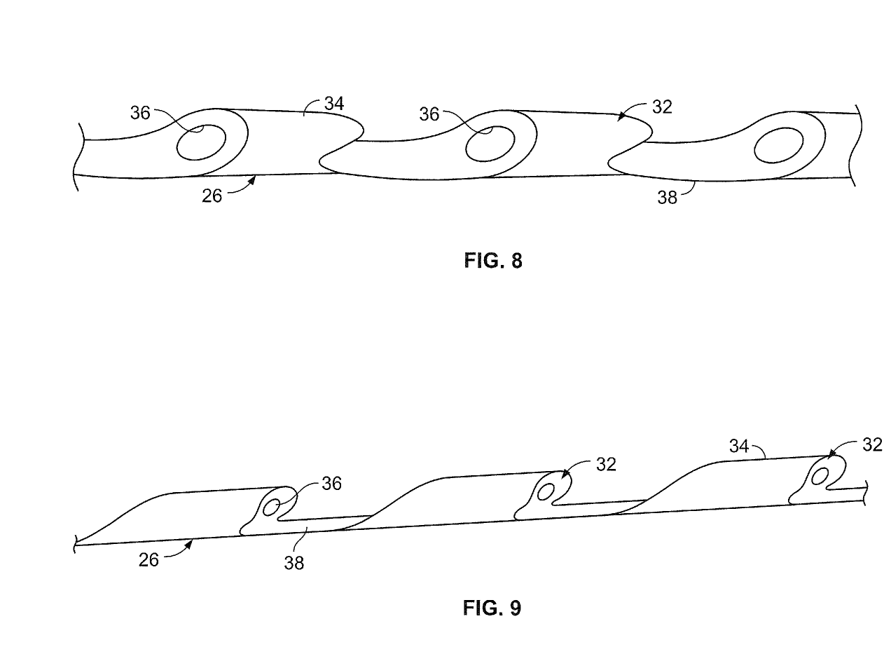

Each tissue engaging element 26 has an outer surface 34 that contacts tissue. Importantly, each outer surface 34 provides contact, specifically a zone of contact, between the suture assembly 10 and tissue. The tissue engaging elements 26 may have substantially identical configurations, or, as shown in FIGS. 1A and 1B, may have configurations dictated by location of the tissue engaging elements 26. FIG. 1A shows the tissue engaging element 26 immediately adjacent the first filament 20 with an outer surface 34 defining a profile 28 that identifies a slope that inclines from the first filament 20 towards the second member 14. In substantially similar fashion, the tissue engaging element 26 immediately adjacent a third filament 30 has an outer surface 34 defining a profile 32 that identifies a slope that declines towards the third filament 30. The profiles 32 of the tissue engaging elements 26 can have different configurations, such as those shown in FIGS. 8 and 9. The profiles 32 can be shaped to encourage tissue migration and growth into the interior surfaces 36 of the tissue engaging elements 26. This increases the tissue holding capability of the suture assembly 10.

The third filament 30 can be constructed substantially similarly to the first filament 20 and the second filament 24. In an exemplary embodiment, the third filament 30 is about 15 cm long. In both cases, the profiles 28 or 32 approach a filament 20 or 30, respectively. The third filament 30 connects the tubular body 22 to the second member 14. In some embodiments, a connector having a length of about 6 mm, a width of about 0.9 mm and a thickness of about 0.13 mm joins the third filament 30 to the second member 14. The profiles 28 and 32 encourage intended healing of tissue, and provide a substantially continuous transition between the tissue engaging elements 26 and the filaments 20, 24 or 30.

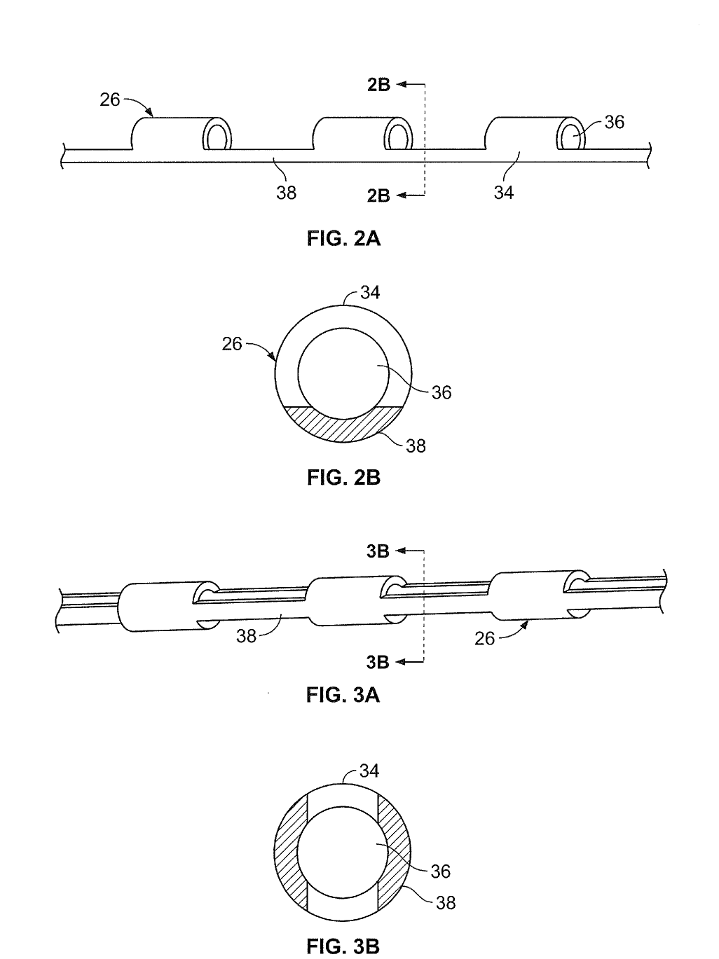

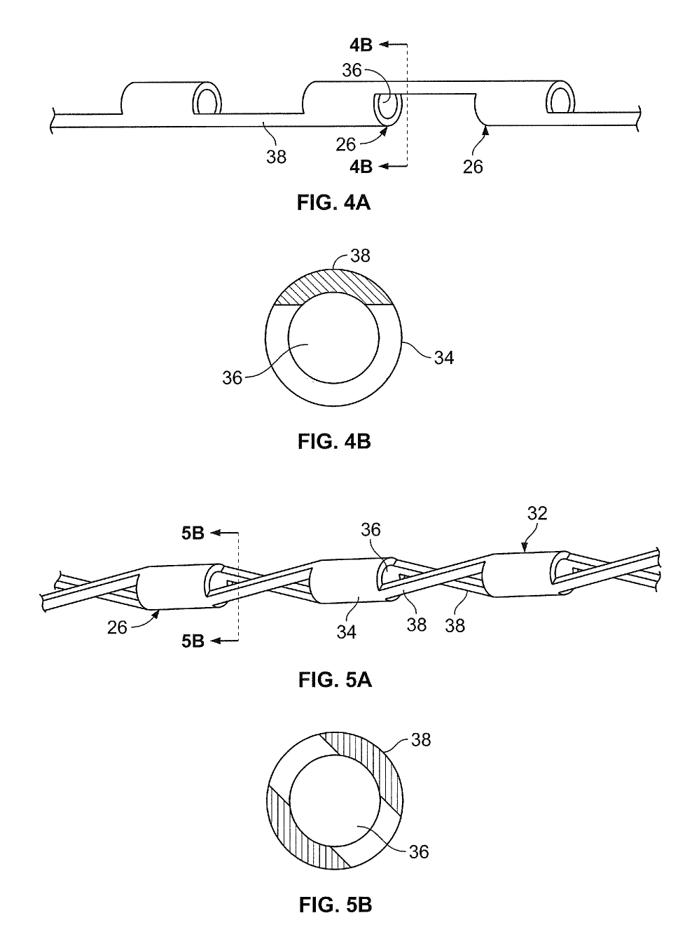

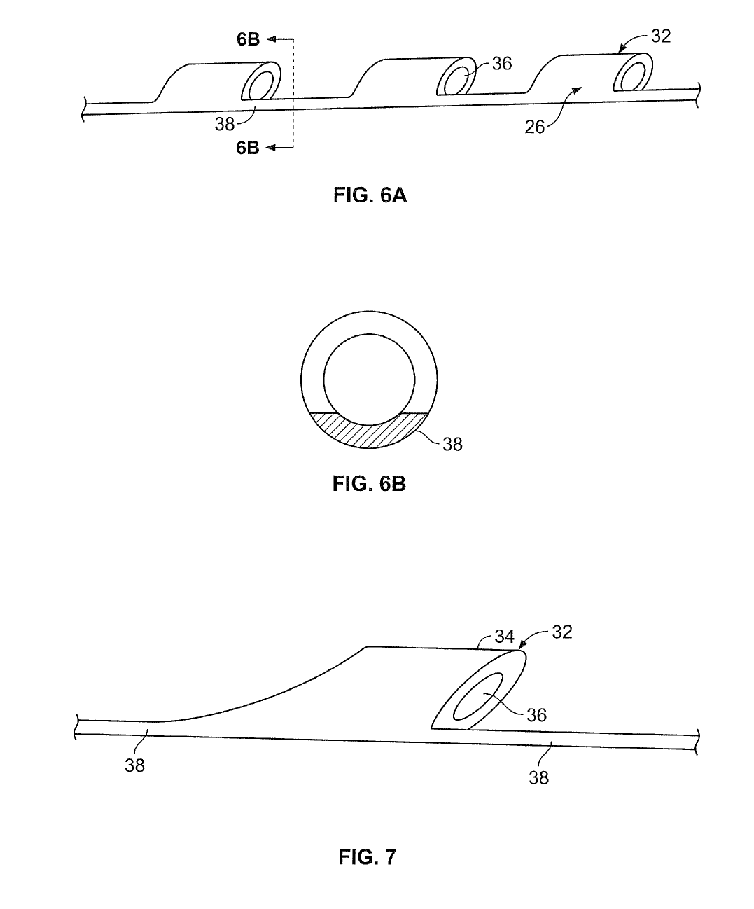

FIGS. 2A through 6B show different constructions of the tissue engaging elements 26 on the tubular body 22. While each pair of FIGS. 2A through 6B displays only one construction of tissue engaging elements 22, it is possible that any mixture of constructions of tissue engaging elements 22 may be disposed on a given tubular body 22. For example, one tubular body 22 may possess tissue engaging elements 26 having the construction of FIG. 2A and tissue engaging elements 26 having the construction of FIG. 6A.

If the tubular body 22 is hollow, the tissue engaging elements 26 on the tubular body 22 will be hollow as well. With a hollow tubular body 22, each tissue engaging element 26 has an interior surface 36 opposite to its outer surface 34. In an exemplary embodiment, the interior surface 36 is defined by a diameter of about 0.50 mm, and a thickness between the outer surface 34 and the interior surface 34 is about 0.25 mm. Of course, if the tubular body 22 is solid, the tissue engaging elements 26 will be solid as well and will not have an interior surface 36. Each interior surface 36 is formed to accept tissue growth.

The tissue engaging elements 26 are formed from the tubular body 22 leaving the tissue engaging elements 26 joined and separated by supporting members 38. Adjacent tissue engaging members 26 are separated by a supporting member 38. In an exemplary embodiment, the supporting member 38 has a thickness of about 0.12 mm and a length of about 5.0 mm. Thus, zones of contact between the suture assembly 10 and tissue provided by the tissue engaging members 26 also are separated by a supporting member 38. Tissue can grow into the interior surfaces 36 as well as into the spaces between the tissue engaging elements 26. Each location of potential tissue growth along the suture assembly 10 provides additional zones of contact between the suture assembly 10 and tissue. These multiple points of contact between the suture assembly 10 and tissue increase integrity of tissue retention, i.e. holding tissue in place, offered by the suture assembly 10.

In some embodiments, a device, such as a trocar, a cylinder that may be removed after suture installation or that may be allowed to dissolve naturally, and the like, may be interested into the interior surfaces 36 to support the tissue engaging elements 26, such as during installation of the suture assembly 10 to tissue and thereby reduce the likelihood of collapse of the tissue engaging elements 26.

In other embodiments, appropriate portions, such as the outer surfaces 34, the interior surfaces 36, the supporting members 38 or subsets of these items, of the tubular body 22 are provided with a compound, such as a drug and the like, that encourages healing. In yet other embodiments, the compound may be a therapeutic drug thereby allowing the suture assembly 10 to provide a method of drug delivery.

The compound may be added to the suture assembly 10 by any suitable technique, such as coating and the like, such that the compound is disposed on at least one of the outer surfaces 34, the interior surfaces 36, and the supporting members 38. The compound may comprise a biocompatable polymer or other additives to further improve faster healing, to promote collagen formation, or to perform another desired operation. The additives used may include, but are not limited to, fatty acids, salts, esters, vitamins (e.g., vitamin C), minerals (e.g., zinc, copper), growth factors (e.g., collagen, fibroblasts, growth factors), antibiotics (e.g., rapamycin) and the like.

One way of applying the compound to the appropriate portions of the tubular body 22 is a coating process described generally as follows. The compound is dissolved or suspended in a volatile organic liquid. Then, that liquid is applied in the form of a liquid coating to the appropriate portions of the tubular body 22. This liquid coating may be applied by dip coating, bushing, wiping, drip coating, spray coating or by using a coating/filling head. Appropriate portions of the tubular body 22 may be dip coated in a batch process by winding the tubular body 22 on a frame and immersing the frame into a coating solution. Alternatively, in a continuous process, the tubular body 22 is passed under tension into a dip tank, and then passed through a drying tunnel. A similar method is described in U.S. Pat. No. 3,982,543, the disclosure of which is incorporated herein in its entirety. In such a continuous dip coating process, tubular bodies 22 can be coated at a rate of about 45-60 feet per minute. Another means of coating a tubular body 22 is to drip coat using a syringe pump that applies the coating to a moving tubular body 22. Tubular bodies 22 can be drip coated at a rate of about 44 meters per minute, An exemplary method is described in U.S. Pat. No. 5,472,702, and the disclosure thereof is incorporated herein in its entirety). Exemplary coating/filling heads are described in U.S. Pat. No. 5,447,100 and the disclosure thereof is incorporated herein in its entirety. Such heads can coat tubular bodies 22 at a rate of about 50 meters per minute.

Further attributes of the suture assembly 10 may become evident in the following discussion of an exemplary utilization of the suture assembly 10. The following method begins with a wound formed on a portion of tissue.

To close the wound, the proximal end 16 of the first member 12 is inserted into the tissue adjacent the wound. The first member 12 is moved with respect to the tissue such that the tubular body 22 is positioned adjacent the wound bringing the outer surfaces 34 of the tissue engaging elements 26 into contact with tissue. In some embodiments of this method, a sleeve, not shown, is provided around the tubular body 22 to reduce drag of the tubular body 22 with respect to the tissue.

The first member 12 is moved with respect to the tissue until desired contact between the tissue engaging elements 26 and the tissue is achieved. If a sleeve is included, the sleeve is removed after the tissue engaging elements 26 have reached the desired position with respect to the tissue. Once the tissue engaging elements 26 are in the desired position with respect to the tissue, multiple zones of contact between the tissue and the suture assembly 10 and the tissue have been achieved. Upon consideration of the number and distribution or arrangement of tissue engaging elements 26 shown in FIGS. 1A and 1B, stability and tissue holding ability of the suture assembly 10 can be appreciated. The second member 14 is moved with respect to the first member 12 such that the suture assembly 10 is fixed, i.e. tied down, with respect to the tissue. The distal end 18 of the first member 12 is removed from the first filament 20, and the second member 14 is removed from the third filament 30. In some methods, the first member 12 is removed before the suture assembly 10 is tied down.

What is claimed is:

1. A suture assembly for use with tissue, the suture assembly comprising:
a) a tubular body;
b) a plurality of tissue engaging elements formed from the tubular body;
c) an outer surface disposed on each tissue engaging element contacting the tissue;
d) an interior surface disposed on each tissue engaging element for accepting tissue growth; and
e) a supporting member joining and separating two tissue engaging elements.

2. A suture assembly as defined in claim 1 further comprising:
f) a filament connected with the tubular body; and
g) a profile defined by the outer surface approaching the filament.

3. A suture assembly as defined in claim 1 further comprising:
f) a compound disposed on at least one of the outer surface, the interior surface and the supporting member.

4. A suture assembly as defined in claim 1 wherein the tubular body has a length, and wherein the plurality of tissue engaging elements are arranged along the length.

5. A suture assembly for use with tissue, the suture assembly comprising:
a) a tubular body;
b) a plurality of tissue engaging elements formed from the tubular body and arranged along a length of the tubular body;
c) an outer surface disposed on each tissue engaging element contacting the tissue; and
d) an interior surface disposed on each tissue engaging element for accepting tissue growth.

6. A suture assembly as defined in claim 5 further comprising:
e) a filament connected with the tubular body; and
f) a profile defined by the outer surface approaching the filament.

7. A suture assembly as defined in claim 5 further comprising
e) a supporting member joining and separating two tissue engaging elements.

8. A suture assembly as defined in claim 7 further comprising:
e) a compound disposed on at least one of the supporting member, the outer surface and the interior surface.

9. A suture assembly for use with tissue, the suture assembly comprising:
a) a tubular body;
b) a plurality of tissue engaging elements formed from the tubular body;
c) an outer surface disposed on each tissue engaging element contacting the tissue; and
d) a supporting member joining and separating two tissue engaging elements.

10. A suture assembly as defined in claim 9 wherein the tubular body has a length and the plurality of tissue engaging elements are arranged along the length.

11. A suture assembly as defined in claim 9 further comprising:
e) a filament connected with the tubular body; and
f) a profile defined by at least one of the tissue engaging elements approaches the filament.

12. A suture assembly as defined in claim 9 further comprising:
e) a compound disposed on at least one of the plurality of tissue engaging members and the supporting member.
